(19) United States
(12) Patent Application Publication
(10) Pub. No.: US 2026/0271896 A1
(43) Pub. Date: Sep. 17, 2026

(54) APPARATUS AND METHOD FOR SUPPLYING SUSTENANCE TO INSECTS

(71) Applicant: Alpha-Protein GmbH, Bruchsal (DE)

(72) Inventor: Rohi Shalati, Karlsruhe (DE)

(21) Appl. No.: 18/872,913

(22) PCT Filed: Apr. 18, 2023
(86) PCT No.: PCT/DE2023/100278

§ 371 Date: Dec. 9, 2024

(30) Foreign Application Priority Data
Jun. 7, 2022 (DE) 10 2022 114 308.2

(51) Int. Cl. A01K 67/362; A01K 67/368

(52) U.S. Cl. CPC A01K 67/362; A01K 67/368

(57) ABSTRACT

The invention relates to an apparatus and a method for providing for insects. Producing cost-effective, protein-rich and sustainable sources for the food and feed industries is one of the challenges of the new millennium, especially given the world's ever-growing population. It is the object of the invention presented here to increase the efficiency and ultimately the yield in the rearing of insects. The invention achieves this by means of an apparatus for providing for insects, comprising at least one vertical transport device and at least one horizontal transport device, wherein the at least one container can be moved in the vertical direction by means of the vertical transport device and in the horizontal direction by means of the horizontal transport device, and comprises a supply device with which a container content of the container and/or the container itself can be supplied. The apparatus according to the invention is characterized in that the horizontal transport device has a measuring device with which properties of the container that can be moved horizontally by means of the horizontal transport device and/or the container contents can be determined before, during and/or after the horizontal transport along a path of the horizontal transport device.

### REFERENCE TO PENDING PRIOR PATENT APPLICATIONS

This patent application is a 371 national stage entry of pending prior International (PCT) Patent Application No. PCT/DE2023/100278, filed 18 Apr. 2023 by Alpha-Protein GmbH for APPARATUS AND METHOD FOR SUPPLYING SUSTENANCE TO INSECTS, which patent application, in turn, claims benefit of German Patent Application No. 10 2022 114 308.2, filed 7 Jun. 2022.

The two (2) above-identified patent applications are hereby incorporated by reference.

### FIELD OF THE INVENTION

The invention relates to an apparatus for providing for insects, comprising at least one vertical transport device and at least one horizontal transport device, wherein the at least one container can be moved in the vertical direction by means of the vertical transport device and in the horizontal direction by means of the horizontal transport device, and comprises a supply device with which a container content of the container and/or the containers can be supplied. Furthermore, the invention relates to a method for providing for insects and the use of the apparatus for providing for insects.

### BACKGROUND OF THE INVENTION

The production of cost-effective, protein-rich and sustainable sources for the food and feed industries represents one of the major challenges and necessities of the new millennium, especially in view of the rapidly increasing population of the world. The rearing of insects, especially mealworms, has proven to be an innovative approach that can provide a cost-effective and sustainable source of protein and be used in both the food and feed industries.

Various apparatuses and methods for rearing such insects are known from the state of the art. In particular, the German patent DE 10 2019 121 102 B3 discloses an apparatus of the type mentioned above and a method for rearing, in particular, insects. The apparatus comprises at least one stack of boxes located on a chassis and a portal, wherein the portal has at least one supply module, one cleaning module, one vertical transporter upwards, one vertical transporter downwards, one transverse transporter on the top, and one transverse transporter on the bottom. Furthermore, the apparatus has a guide system on which the portal can move across a stack of boxes and which has a conveyor system with which boxes can be conveyed to and from the stacks. Finally, the portal can be used to cyclically rearrange, supply and clean the boxes within the stacks of boxes, and boxes can also be moved out of and into the stack of boxes through the portal.

Also known from the Chinese utility model CN 209403339 U is a fully automatic destacking machine in the field of aquaculture equipment, with which boxes can be stacked and destacked. Said equipment consists of a buffer station, a lifting station, a destacking station and a conveyor station.

Furthermore, the Chinese utility model CN 209824895 U discloses a feeding apparatus for use in rearing insects. The feeding apparatus comprises a support frame, a feeding box and a screw conveyor device with a conveyor screw, wherein the conveyor screw is installed on the support frame in the middle, the feeding box is attached to the top of the support frame and the top of the feeding box is firmly connected to a chopping mechanism.

The use of the chopping mechanism is intended to chop the material to be fed appropriately before it is added so as not to harm the insects when the feed is added and to make it easier for the insects to ingest the feed.

An apparatus is also known from the Chinese patent application CN 107691384 A, with which the rearing of mealworms can be carried out automatically. The apparatus comprises an outer frame, an inner frame, a lifting system, a telescopic system, a feeding system, a scatter feeding system and a raceway system.

Furthermore, from US2016/0066552 A1, a method and a system for rearing insects using a large number of individual boxes is previously known, in which at least part of each box is filled with a substrate containing feed and immature phases of insects.

### SUMMARY OF THE INVENTION

Based on this state of the art, it is the object of the present invention to increase the efficiency and ultimately the yield when rearing insects. This object is achieved with an apparatus according to independent claim 1. Furthermore, this object is achieved by a method according to the independent method claim 24 and by using an apparatus according to the invention according to the independent use claims 30 to 33. Advantageous embodiments of the invention can be found in the dependent claims.

In a first aspect, the invention relates to an apparatus for providing for insects. Said apparatus comprises at least one vertical transport device and at least one horizontal transport device, wherein at least one container contains a container content which preferably comprises insects, and the at least one container is movable in the vertical direction by means of the vertical transport device and in the horizontal direction by means of the horizontal transport device. For example, several, preferably two, container stacks standing on pallets can be simultaneously rearranged according to the paternoster principle next to one another and/or one after the other by the horizontal transport device and vertical transport device.

In addition, the apparatus has a supply device with which the container contents, in particular, the insects located in the container contents, can be provided for.

The apparatus according to the invention is characterized in that it has at least one measuring device. By means of this measuring device, the apparatus can detect properties of the containers that can be moved horizontally by means of the horizontal transport device and/or of the container contents before, during and/or after the horizontal transport along a path of the horizontal transport device. It is also conceivable that data is collected over a defined period of time.

With the apparatus according to the invention it can be achieved that during the transport of the at least one container by the horizontal transport device, the properties of said container and/or the container contents can be detected before, during and/or after transport, without a further step being necessary for this, whereby the conveying rate and the efficiency of providing for insects, for example, can be increased. As a result, an increased and, through increasing knowledge, optimized rearing of the container contents, in particular, of insects, can be achieved.

By associating the measuring device with the horizontal transport device, a more compact and comparatively space-saving design can be achieved. In particular, an integration of the measuring device into the horizontal transport device is conceivable. In this case, the measuring time can be extended by moving the measuring device with the horizontal transport device or the transport time can be used for the measurement.

According to an alternative embodiment, the at least one measuring device is arranged along the path of the horizontal transport device or before or after it.

It is conceivable that one measuring device each is arranged before, along or after the path of the horizontal transport device. This can ensure, for example, that properties of the container and/or its container contents can be recorded at any time during transport by the horizontal transport device.

According to a further advantageous embodiment, the measuring device may have a weighing appliance for determining the weight of the container and/or its container contents. By determining the weight of the respective container, for example, a mass change curve of the containers, or more precisely of the container contents over time, can be determined and from this, in particular, the mass change curve of each container content can be created on the basis of the measured values at different measurement times.

By determining the weight and the respective weight change of the container contents, the exact amount of feed required for further rearing can be determined. For insects, for example, the optimal amount of food for ad libitum feeding can be determined for the respective stage of the insects in the containers and excess feeding can be prevented. The overall system is therefore more efficient and the optimal harvest time can be determined more effectively. Even incorrectly filled boxes can be identified at this point and removed from the apparatus.

Particularly preferably, the weighing appliance consists of load cells. It is conceivable that the container is picked up by the weighing appliance on several sides, edges or several corners by the weighing cells or the pickup devices associated with them, so that during the picking up of the container it can also be determined whether this container has any damage or deformation. Furthermore, an inhomogeneous distribution of the container contents can be detected by picking up the containers on several sides or several corners.

In order to be able to clearly associate the properties determined by means of the measuring device, in particular, the weight of the container and/or its contents, with the respective container, container stack and/or pallet with containers and/or container stack, the apparatus provides in a further advantageous embodiment that the respective container has at least one marking element with which the respective container is marked and can be clearly identified. It is also conceivable to mark a container stack and/or a pallet with containers and/or container stacks with a marking element. Thus, the properties of the containers, container stacks and/or pallets with containers and/or container stacks and/or the container contents determined by means of the measuring device can each be clearly associated with a container, a container stack and/or a pallet of containers and/or container stacks.

Preferably, the marking element is a marking element that can be read with a device, in particular, an RFID transponder, a QR code, a combination thereof or another marking element that can be read with a detecting device. Particularly advantageously, the horizontal transport device has a device for reading and identifying the marking element with the aid of which a detection and/or identification of a container, a container stack and/or a pallet with containers and/or container stacks is ensured. Thus, for example, the properties determined by the measuring device before, during or after the horizontal transport of at least one container by the horizontal transport device can be associated with the respective container, container stack and/or pallet of containers and/or container stacks clearly determined by the detecting device.

In an alternative embodiment, only the pallet on which the containers and/or container stacks are located is detected and the respective containers themselves are identified by their position in the stack, wherein the position of the respective container is stored, for example, in a database. According to a further, alternative embodiment, it is conceivable that the determination of the containers, container stacks and/or pallets of containers and/or container stacks takes place via information from a central control system, for example, by means of a material flow control system or by means of such a system or by means of a warehouse management system.

In an advantageous embodiment, the invention also provides that the apparatus has an inactivation module, which optionally also inactivates the insects, optionally using microwaves, preferably arranged on the horizontal transport, with which individual containers can be decontaminated and/or sealed, for example, by foaming the container.

In a further advantageous embodiment, the apparatus has a device for mixing and/or loosening the container contents, in which, for example, the container contents are completely or partially stirred and/or mixed and/or loosened. This results in a homogenization of the container contents, which enables more optimal nutrient absorption and increased respiratory activity of the container contents, thus preventing anaerobic zones.

In order to be able to take samples from a container or from its container contents, the invention provides, in a further advantageous embodiment, that a sampling module is associated with the horizontal transport device. In a preferred embodiment, said sampling module is associated with the horizontal transport device such that samples of the container contents can be taken before, during and/or after the horizontal transport of the at least one container. This means that, for example, samples can be taken and examined at random.

However, it is also conceivable that no horizontal transport takes place, but rather that only one container of a container stack is lifted by the horizontal transport device and/or the sampling module and a sample is taken from the container or the container contents.

In a further advantageous embodiment, the invention provides for the apparatus according to the invention to have at least one further measuring device arranged along the path of the horizontal transport device. By means of this additional measuring device, further measurement data on the condition of the containers and/or the container contents can be determined.

Preferably, the further measuring device has at least one sensor for determining the fill level of a container and/or a sensor for detecting the surface profile of the container contents, preferably the entire surface profile. This can be an infrared sensor or another suitable optical sensor which can detect when a defined height or the height of the container and its contents is exceeded, for example, based on the breaking of a generated light barrier.

This makes it, in particular, possible to determine whether the container is deformed or overfilled and/or there is a possible heterogeneity of the respective fill levels or an inhomogeneous filling distribution of the containers. In addition, by using different light sensors, different layers of the container contents can be penetrated in order to determine, for example, the value of a specific content.

Furthermore, by combining the determined fill levels of a container and the weight data that can also be detected, conclusions can be drawn about the density of the container contents and thus, for example, about the amount of insects or the volume of living biomass, so that the volume and weight of the container contents can be determined by measurement and data comparison.

Particularly preferably, the at least one further measuring device has at least one optical measuring device, in particular, a camera for detecting the state of the container and/or the container contents. By means of the optical measuring device, in particular the camera, it can be determined, among other things, whether there is a malfunction of the supply device due to over- or under-feeding or whether there are any foreign bodies in the container.

It is also conceivable that a sensor device, such as a sensor cube, which records all the required parameters (humidity, temperature, substrate height, photographic images, etc.), which sensor device is inserted at the beginning of the rearranging process or of the horizontal transport and is then removed again, and reports the data to the apparatus via wireless communication, and has a plurality of sensors or devices for recording any relevant data, in particular the humidity, temperature, substrate height, optical measurement data, etc., which is added into the container during rearranging or supplying by the apparatus and then removed again before the container exits the apparatus and the data collected in the container are transmitted wirelessly, for example, to the apparatus.

Empty or dead containers as well as any infestation with parasites or other pathogens, such as fungi, can also be detected in this way. But also the feed consumption and the growth process of, for example, insects, can be detected by means of the optical measuring device, in particular the camera. This is done, for example, by determining the number of insects, the amount of food and any residues of the insects, such as feces or exuviae, from the data recorded by the optical measuring device, in particular the camera. Furthermore, it is conceivable that the optical measuring device, in particular the camera, records both images and video sequences. The use of special cameras, such as infrared cameras, is also conceivable in this context.

Furthermore, in an again advantageous embodiment of the invention, the apparatus provides that the at least one further measuring device has at least one sensor for determining the humidity prevailing within the container. This makes it possible to determine, among other things, whether too much water or too much moist food has been added to the insects in the container by the supply device. The increase in humidity inside the container may also indicate a leak in a water pipe on the apparatus or provisioning system, which can be detected with the humidity sensor.

Furthermore, in a further advantageous embodiment, the apparatus according to the invention has at least one sensor on the at least one further measuring device for determining the surface temperature within the container and/or the surface of the container contents and/or the temperature prevailing in the container contents.

By detecting the temperature prevailing inside the container, it is possible to determine, among other things, whether the temperature is optimal for the growth and survival of the insects. In addition, an increased or decreased temperature can also be an indicator of the presence of a problem in the container, such as a disturbed microclimate due to a lack of air circulation. For example, an increased temperature could indicate contamination of the container contents, in particular of insects, whereas a temperature that is too low could indicate the death of the container contents, in particular the insects, or hypothermia due to possibly faulty climate control and incorrectly set air conditioning. By detecting the temperature of the container and/or the container contents, in particular the insects inside the container, it is possible to react to a temperature deviating from an optimal value.

According to a further, again advantageous further embodiment of the subject matter of the invention, the invention comprises an additional “box contents mixing or loosening module” to ensure a homogeneous distribution of the various components of the container contents.

Advantageously, the supply device may be arranged along the path of the horizontal transport device. Thus, during the horizontal transport of the container by the horizontal transport device, the container or the container contents can be supplied with any supply materials in the same step. This can ensure an increased or more efficient supplying of the container or its contents.

Preferably, the supply device has at least one supply module for dispensing a supply material, in particular feed, substrate, water, insects, worms, plants, algae or other organic substances. Due to this advantageous modular design of the supply device, the respective needs of the container or container contents that are transported horizontally by the horizontal transport device and are to be supplied with supply material can be individually addressed with the appropriate or required materials. This ensures that the optimal conditions are always created for the container contents, especially for the growth and survival of insects.

In order to be able to remove any residues or contaminants or other foreign bodies from the containers, a further advantageous embodiment of the apparatus provides that the supply device has a cleaning module for cleaning the container. Vacuuming to remove any foreign substances and/or contaminants and/or residues is also conceivable in this context.

In order that the apparatus can be individually adapted to any existing spatial conditions, in particular, with regard to the size and/or type of containers, the container stack and/or the pallet with containers and/or container stacks as well as the container contents, the apparatus is constructed from a number of modules in a further advantageous embodiment.

This is achieved in a preferred embodiment in that the modules are composed of a lower frame, a main frame, a number of connecting pieces and an upper frame. The horizontal transport device and the vertical transport device can be attached to the main frame or the horizontal transport device can be attached to the upper frame and/or lower frame, wherein the supply device may be arranged on the upper frame. For example, there may be any conveying devices on the lower frame, for example a roller conveyor, with which a container, a container stack and/or a pallet with containers and/or container stack can be conveyed to the apparatus, in particular, to the vertical transport device. The connecting pieces can be used to connect the lower frame, the main frame and the upper frame as well as to connect one main frame to another main frame.

In an again advantageous embodiment, the invention provides for the apparatus to have an upstream and/or downstream buffer area at which at least one container, a container stack and/or a pallet with containers and/or container stacks can be placed and/or removed. Preferably, the buffer area has contour scanners which check the exact position or the exact fit of the containers in a container stack or of the container stack on a pallet and, if necessary, bring about the exact, congruent alignment of the containers or the container stack with respect to one another by means of stack clamps or other suitable means.

For example, palletized containers or container stacks from a warehouse logistics facility can be placed on this upstream buffer area, which in turn can be transported from this buffer area in the direction of, in particular to, the vertical transport device, wherein they can be rearranged by the vertical transport device and the horizontal transport device in a paternostric principle.

For this purpose, in an advantageous embodiment, the apparatus can have at least one positioning unit with which the exact positioning of a pallet with containers and/or container stacks or a container stack or at least one container within the apparatus can be ensured. Subsequently, the containers that have been rearranged by means of the vertical transport device and the horizontal transport device and that have been supplied with supply material by the supply device during horizontal transport can be discharged from the apparatus into a downstream buffer area. In a preferred embodiment, the container, the container stack and/or the pallet with containers and/or container stacks can be conveyed into and out of the apparatus by a conveyor system. The containers, container stacks and containers supplied and discharged from the apparatus can be

removed again from said downstream buffer area by a warehouse logistics device.

Alternatively, the apparatus may also have a device for connecting to a conveyor system, with which the apparatus can be connected to, for example, a roller conveyor, with which incoming or discharged containers, container stacks and/or pallets with containers and/or container stacks can be transported to or away from the apparatus.

Furthermore, in a second aspect, the invention relates to a method for supplying insects, in particular by means of a device according to the invention. Said method is characterized by the properties of a container and/or a container content being determined before, during and/or after a horizontal transport of the container and the properties thus determined are stored as empirical data and/or used to directly adapt or optimize the supply of the container content, in particular the insects. The properties of the containers and/or the container contents can be determined, for example, by the measuring device arranged on the apparatus, which is preferably arranged along the path of the horizontal transport device, and/or by the further measuring devices arranged along the path of the horizontal transport device. This makes it possible, for example, to determine whether the addition of any supply materials during horizontal transport by the horizontal transport device was carried out correctly. For example, over-or under-feeding can be determined by determining the properties before and after horizontal transport.

In a further advantageous embodiment of the method, the invention provides that the container contents are supplied with supply materials during horizontal transport.

Depending on the specific properties of the container and/or the container contents, the supply of supply materials can be carried out by, for example, at least one supply device. For example, only a supply material, in particular food, substrate, water, insects or insect eggs, may be added to the container, which is conducive to optimal growth promotion.

In order to increase the efficiency of the supply of any containers during horizontal transport, in particular by a horizontal transport device, the invention provides in a further advantageous embodiment of the method that several containers are transported horizontally at the same time.

In order to ensure that the containers or the container contents, in particular the insects located therein, can be optimally supplied with supply materials, the invention provides in a further advantageous embodiment of the method that the properties of the containers and/or container contents determined during horizontal transport are compared with the empirical data already stored. By comparing the properties determined during horizontal transport with the empirical data already stored, machine learning can be carried out in a further advantageous embodiment, whereby the condition of the containers and/or the container contents can be classified, evaluated and supplied more effectively.

In a further advantageous embodiment, the invention provides for the method that actions are carried out on the basis of the compared data, for example, that a container is discharged or that supply material is added. It is also conceivable that the container is cleaned, for example by a cleaning module that may be present.

Finally, in further aspects, the invention provides for the use of a device according to the invention for supplying insects, preferably in or on a high-bay warehouse, plants, or organic substances in the maturation and/or fermentation process, in particular fungi, as well as algae.

The invention is then explained in more detail using an embodiment.

### BRIEF DESCRIPTION OF THE DRAWINGS

The drawings show:

FIG. 1: a side view of an embodiment of the apparatus according to the invention, and

FIG. 2: the embodiment according to FIG. 1 in a perspective view.

### DETAILED DESCRIPTION OF THE PREFERRED EMBODIMENTS

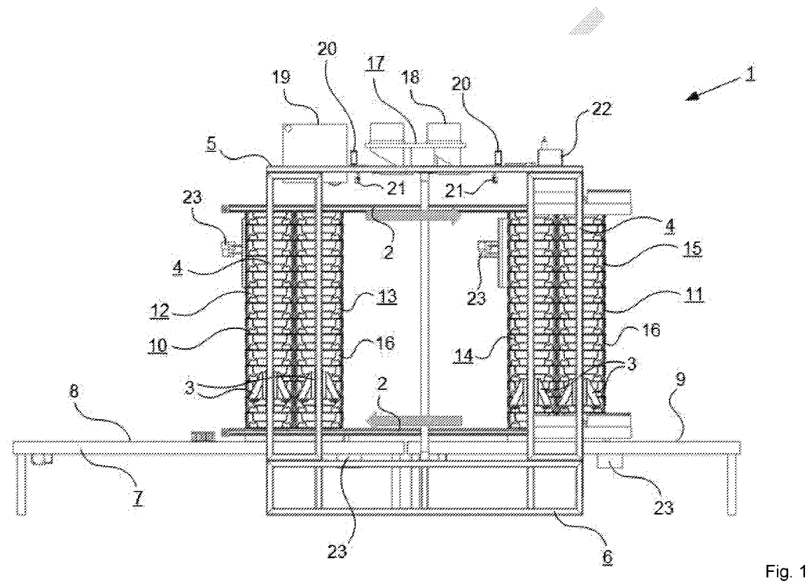

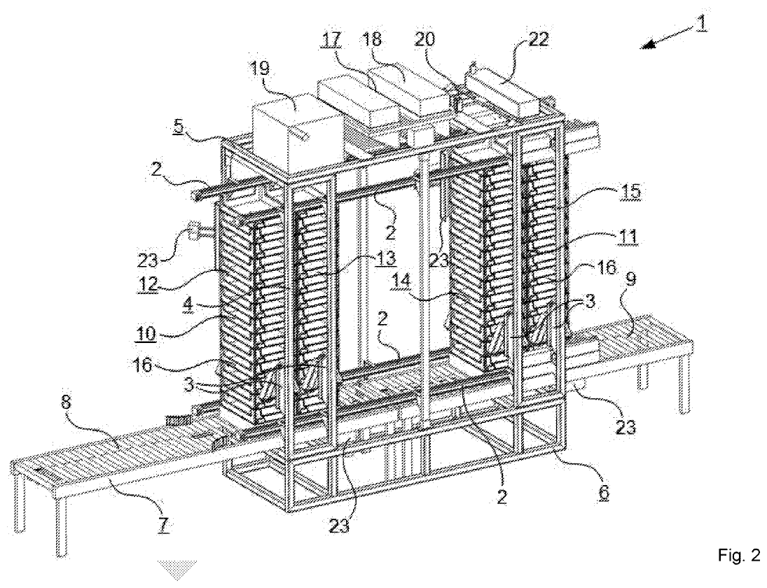

FIG. 1 shows an exemplary embodiment of a device 1 according to the invention in a side view and FIG. 2 shows the embodiment according to FIG. 1 in a perspective view. The apparatus 1 is constructed from modules consisting of a main frame 4, a lower frame 6 and an upper frame 5, which are connected to one another by connecting pieces not shown here. Arranged on the support 6 is a conveyor system 7 with which double container stacks 10, 11 can be conveyed from an upstream buffer area 8 into the apparatus 1 to the vertical transport devices 3.

With the conveyor system 7, the double container stacks 10, 11 rearranged and supplied in the apparatus 1 can subsequently be conveyed out of the apparatus 1 to a downstream buffer area 9 again. The double container stacks 10, 11 can be brought both to the upstream buffer area 8 as well as from the downstream buffer area 9 to and from the apparatus 1 by a warehouse logistics device, for example, a storage and retrieval machine. The double container stacks 10, 11 can, for example, originate from a high-bay warehouse, into which they are transported back by a warehouse logistics device after passing through the supply in the apparatus 1 according to the invention.

After the double container stacks 10, 11 have been conveyed from the upstream buffer area 8 into the apparatus or to the vertical transport device 3 by the conveyor system 7, the containers 16, i.e., both the containers 16 of the left container stack 12, as well as the left double container stack 10 and the right container stack 13 of the left double container stack 10, are lifted by the vertical transport device 3 associated with the left double container stack 10, so that the horizontal transport device 2 arranged on the upper frame 5 can pick them up and then transport them horizontally in the direction of the right double container stack 11. Conversely, the containers 16 of the right double container stack 11 can also be transported from right to left.

The rearranging of the two double container stacks 10,11 shown can be done according to a paternoster principle. In this case, the upper containers 16 of the left double container stack 10, i.e., the containers 16 of the left and right container stacks 12 and 13, are picked up by the horizontal transport device 2, which is arranged on the upper frame 5 of the apparatus 1, and transported horizontally along the path of the upper frame 5 to the right double container stack 11.

The direction of movement of the horizontal transport device 2 arranged on the upper frame 5 is indicated by an arrow. At the same time, the right double container stack 11 is lifted by the vertical transport device 3 associated with the right double container stack 11 at the second lowest containers 16, i.e., at the containers 16 of the left and right container stacks 14 and 15, so that the lower two containers 16 of the right double container stack 11 can be transported horizontally along the lower frame 6 to the left double container stack 10 by the horizontal transport device 2 arranged on the lower frame 6.

Here too, the direction of movement of the horizontal transport device 2 arranged on the lower frame 6 is indicated by an arrow along the horizontal transport device 2. In order to be able to insert the containers 16 transported from bottom right to bottom left into the lower area of the left double container stack 10, the vertical transport device 3 associated with the left double container stack 10 lifts the containers 16 of the left double container stack 10 at the lower containers 16 of the double container stack 10, i.e., the containers 16 of the left and right container stacks 12 and 13, whereby the lower containers 16 conveyed from the right double container stack 11 by means of the horizontal transport device 2 arranged below can be arranged under the left double container stack 10 and the left double container stack 10 can in turn be lowered onto them by the vertical transport device 3 associated with the left double container stack 10.

The steps described above are carried out until all containers 16 have been guided at least once by the horizontal transport device 2 arranged on the upper frame 5 along the devices arranged on the upper frame 5, here the supply device 17 and the cleaning module 19, and all containers 16 of the two double container stacks 10, 11 were able to be supplied by the apparatus 1.

The correct positioning of the containers 16 or the double container stack 10, 11 within the apparatus 1 for carrying out a rearrangement or for horizontal and vertical transport is ensured by the positioning units 23 arranged at different locations. The positioning units 23 can be used to identify the position of a respective container 16 or a double container stack 10, 11, so that in the event of a deviation from an ideal position for transport or rearrangement, a corresponding repositioning can be carried out. For example, in the case of an improperly inserted double container stack 10, 11 or a container stack 12, 13, 14, 15 of a double container stack 10, 11 into the apparatus 1 by the conveyor system 7, wherein the improper insertion can be detected by the positioning units 23, the double container stack 10 or 11 or the respective container stack 12, 13, 14, 15 can be repositioned by the conveyor system 7 and/or the positioning unit 23 itself. It is also conceivable that the positioning unit 23 ensures from the outset that the double container stacks 10, 11 conveyed by the conveyor system 7 or the respective container stacks 12, 13, 14, 15 of the double container stacks 10, 11 are correctly positioned. This can be done, for example, by driving the double container stacks 10, 11 transported by the conveyor system 7 or a container stack 12, 13, 14, 15 of the double container stacks 10, 11 against a device (not shown here), in particular a type of “stopper”, which ensures the correct positioning of the double container stacks 10, 11 or the container stacks 12, 13, 14, 15 of the double container stacks 10, 11.

Along the path of the horizontal transport device 2 arranged on the upper frame 5, further measuring devices may be located on the horizontal transport devices 2, in addition to the fill level sensor 21 shown here, which serves to determine the fill level of the container contents of a container 16 and/or to determine the surface structure of the container contents, wherein this can be done for example by a three-dimensional measurement of the surface of the container contents, which can be used to determine any further properties of the containers 16 transported horizontally by the horizontal transport device 2. In the exemplary embodiment shown in FIGS. 1 and 2, a further measuring device in the form of a camera 20 is located both after the cleaning module 19 arranged on the upper frame 5 and after the supply device 17.

The cleaning module 19 can be used to clean the containers 16 of the left double container stack 10 to be transported by the horizontal transport device 2 arranged on the upper frame 5, and data from the container 16 and/or the container contents can be collected before, after and/or during the cleaning process using measuring devices, for example the cameras 20 and the fill level sensor 21. The data thus collected can be used together with further data collected from the containers 16 and/or the container contents, which can be obtained, for example, by determining the temperature, humidity or weight of the container 16, to determine the condition of the container 16 or the container contents.

After the containers 16 have been cleaned by means of the cleaning module 19 and data have been collected by, for example, the camera 20, the containers 16 are transported horizontally along the upper frame 5 in the direction of the right double container stack 11 by the horizontal transport device 5 arranged on the upper frame 5. During transport, the containers 16 can be supplied with supply materials by the supply device 17. Supply materials may include, among others, feed, substrate or water, and, in particular, in the case of insect rearing, also insect eggs or insects as well as other suitable materials necessary for growth or used to promote growth and animal health. For this purpose, the supply device 17 has different supply modules 18, each of which contains one or more supply materials and can dispense these in portions into the container 16 that can be transported horizontally by the horizontal transport device 2. Furthermore, the apparatus 1 of the embodiment has a sampling module 22, which is also arranged on the upper frame 5. Said sampling module can be used to take samples from the container 16 or the container contents and use them for analysis either immediately or at a later time.

Thus, a device and a method for supplying insects is disclosed above, with which an increase in efficiency and ultimately in the yield in the rearing of insects, in particular, is achieved by the horizontal transport device having a measuring device with which properties of the containers that can be moved horizontally by means of the horizontal transport device and/or the container contents can be detected during horizontal transport along a path of the horizontal transport device and thus data can be generated for further optimization steps.

### List of Reference Signs

1 Apparatus
2 Horizontal transport device
3 Vertical transport device
4 Main frame
5 Upper frame
6 Lower frame
7 Conveyor system
8 Upstream buffer area
9 Downstream buffer area
10 Left double container stack
11 Right double container stack
12 Left stack of the left double container stack
13 Right stack of the left double container stack
14 Left stack of the right double container stack
15 Right stack of the right double container stack
16 Containers
17 Supply device
18 Supply module
19 Cleaning module
20 Camera
21 Fill level sensor
22 Sampling module
23 Positioning unit

1.-33. (canceled)

34. An apparatus (1) for providing for insects, comprising at least one vertical transport device (3) and at least one horizontal transport device (2), wherein the at least one container (16) is movable in the vertical direction by means of the vertical transport device (3) and in the horizontal direction by means of the horizontal transport device (2), and comprises a supply device (17) with which a container content of the container (16) and/or the container itself (16) can be supplied,
characterized in that the apparatus has at least one measuring device with which properties of the at least one container (16) that can be moved horizontally by means of the horizontal transport device (2) and/or of the container contents can be determined before, during and/or after the horizontal transport along a path of the horizontal transport device (2), wherein the at least one measuring device is associated with the horizontal transport device (2).

35. The apparatus according to claim 34, characterized in that the at least one measuring device is arranged along the path of the horizontal transport (2) or before or after it.

36. The apparatus according to claim 34, characterized in that the measuring device has at least one weighing appliance for determining the weight of the container (16) and/or its contents, wherein the weighing appliance preferably consists of weighing cells.

37. The apparatus according to claim 34, characterized in that the container (16), a container or double container stack (10, 11, 12, 13, 14, 15) and/or a pallet with at least one container (16) and/or at least one container stack (10, 11, 12, 13, 14, 15) has at least one marking element with which the container (16), the container stack (10, 11, 12, 13, 14, 15) and/or the pallet with at least one container (16) and/or at least one container stack (10, 11, 12, 13, 14, 15) is marked and can be identified wherein the marking element preferably is a marking element that can be read with a device, in particular, an RFID transponder, a QR code, a combination of these and/or another means suitable for identification.

38. The apparatus according to claim 37, characterized in that it has at least one device for reading the marking element and identifying the container (16), the container stack (10, 11, 12, 13, 14, 15) and/or the pallet with containers (16) and/or container stack (10, 11, 12, 13, 14, 15).

39. The apparatus according to claim 34, characterized in that
it has an inactivation module for decontamination and/or sealing of a container (16), which preferably is arranged on the horizontal transport device (2),
and/or it comprises a device for mixing and/or loosening the container contents of a container (16),
and/or it has at least one cleaning module (19) for cleaning the container (16), in particular, for removing residues created during use.

40. The apparatus according to claim 34, characterized in that a sampling module (22) is associated with the horizontal transport device (2), preferably such that samples of the container contents can be taken before, during and/or after the horizontal transport.

41. The apparatus according to claim 34, characterized in that at least one further measuring device is arranged along the path of the horizontal transport device (2), wherein the further measuring device preferably
has at least one sensor for determining the fill level (21) of a container (16) and/or for detecting the surface profile, in particular the entire surface profile, of the container contents,
and/or has at least one optical measuring device, in particular, a camera (20) for detecting the state of the container (16) and/or container contents, in particular, the insects located in the container contents,
and/or has at least one sensor for determining the humidity prevailing within the container (11),
and/or has at least one sensor for determining the temperature prevailing within the container.

42. The apparatus according to claim 34, characterized in that the supply device (17) is arranged along the path of the horizontal transport device (2).

43. The apparatus according to claim 34, characterized in that the supply device (17) has at least one supply module (18) for dispensing a supply material, in particular feed, substrate, water and/or insects.

44. The apparatus according to claim 34, characterized in that it is constructed modularly from a number of modules, so that the apparatus (1) can be adapted in particular to the size, number and type of a container (16), a container stack (10, 11, 12, 13, 14, 15) and/or a pallet with containers (16) and/or a container stack (10, 11, 12, 13, 14, 15) as well as the container contents.

45. The apparatus according to claim 34, characterized in that it has an upstream (8) and/or downstream buffer area (9) on which the at least one container (16), a container stack (10, 11, 12, 13, 14, 15) and/or a pallet with at least one container (16) and/or at least one container stack (10, 11, 12, 13, 14, 15) can be placed and/or from which it can be removed.

46. The apparatus according to claim 34, characterized in that it has at least one positioning unit (23) with which a positioning of the at least one container (16), a container stack (10, 11, 12, 13, 14, 15), an individual container within a container stack, and/or a pallet with at least one container (16) and/or at least one container stack (10, 11, 12, 13, 14, 15) within the apparatus (1) is ensured.

47. The apparatus according to claim 34, characterized in that it has a conveyor system (7) for conveying the at least one container (16), a container stack (10, 11, 12, 13, 14, 15) and/or a pallet with at least one container (16) and/or at least one container stack (10, 11, 12, 13, 14, 15) into and out of the apparatus (1) and in that preferably it has a connecting device for connecting to a conveyor system (7), in particular a roller conveyor.

48. A method for providing for insects with an apparatus (1) according to claim 34, characterized in that properties of a container content and/or a container (16) are determined before, during and/or after a horizontal transport of the container (16) and the properties thus determined are stored as empirical data and/or used for the direct adaptation and/or optimization of providing for the container content, in particular the insects.

48. The method according to claim 48, characterized in that the container (16) and/or the container contents are supplied with supply materials before, during and/or after the horizontal transport.

49. The method according to claim 48, characterized in that several containers (16) are transported horizontally at the same time.

50. The method according to claim 48, characterized in that the properties of the container (16) and/or the container contents, in particular the insects, determined during the horizontal transport are compared to the stored empirical data, wherein preferably collected data are used for machine learning.

51. The method according to claim 49, characterized in that, on the basis of the compared data, instructions for action are derived, in particular, that a container (16) is discharged or that supply materials are added.

52. A use of an apparatus (1) according to claim 34 for providing for
insects in or at a high-bay warehouse
and/or plants
and/or algae or fungi
and/or organic substances and mixtures in the maturation process and/or fermentation process.
